(19) United States
(12) Patent Application Publication
(10) Pub. No.: US 2026/0185034 A1
(43) Pub. Date: Jul. 2, 2026

(54) CULTURE PROCESSING DEVICE

(71) Applicant: SHIBUYA CORPORATION, Kanazawa-shi (JP)

(72) Inventors: Kenji YONEDA, Kanazawa-shi (JP); Masaru ISHII, Kanazawa-shi (JP); Naoya DEGUCHI, Kanazawa-shi (JP); Noriyuki MATSUO, Kanazawa-shi (JP); Noriaki NISHIMURA, Kanazawa-shi (JP)

(21) Appl. No.: 19/430,458

(22) Filed: Dec. 23, 2025

(30) Foreign Application Priority Data
Dec. 26, 2024 (JP) 2024-230000

(51) Int. Cl. C12M 1/36; C12M 1/00

(52) U.S. Cl. CPC C12M 41/48; C12M 29/26

(57) ABSTRACT

Culture processing device including processing rooms partitioned to correspond to first to fourth processing processes, a container moving device moving a container from a processing room for an upstream process to a processing room for a downstream process. The culture processing device includes an air conditioning device for supplying cleaned gas from upper parts of the processing rooms and discharging cleaned gas from lower parts of the processing rooms to ventilate. When a container storing a culture different from a culture stored in a precedingly processed container is processed, the container moving device moves the preceding container from the processing room for the upstream process to the processing room for the downstream process. Thereafter, the air conditioning device ventilates the processing room for the upstream process for a predetermined time and, the container moving device carries the container storing the different culture into the processing room for the upstream process.

### BACKGROUND OF THE INVENTION

### Field of the Invention

The present invention relates to a culture processing device and, in particular, relates to a culture processing device that performs, in a plurality of processing rooms partitioned to correspond to a plurality of processing processes, processing corresponding to a process on a container storing a culture.

### Description of the Related Art

Nowadays, it is customary to culture a cell collected from a subject for use in treatment for the subject himself or herself. In order to cope with treatment for a large number of subjects, a culture processing device capable of automatically performing processing required for the culture has been used.

As the culture processing device explained above, there is known a culture processing device including a plurality of processing rooms partitioned to correspond to a plurality of processing processes, processing device provided in the plurality of processing rooms to perform processing corresponding to a process on a container storing a culture, container moving device for moving the container from a processing room for an upstream process to a processing room for a downstream process adjacent to each other, and control device for controlling the processing device and the container moving device (Japanese U.S. Pat. No. 5,706,610 (Patent Literature 1)).

In Patent Literature 1, the container moving device moves the container from the processing room for the upstream process to the processing room for the downstream process to perform a series of processing.

### SUMMARY OF THE INVENTION

However, in Patent Literature 1, since the processing rooms are sterilized to prevent contamination, there is a temporal loss in continuously performing the processing in order to cope with a large number of subjects.

In view of such a problem, the present invention provides a culture processing device capable of efficiently performing processing for a culture while preventing contamination.

Specifically, a culture processing device according to an invention of claim 1 is a culture processing device including: a plurality of processing rooms partitioned to correspond to a plurality of processing processes; processing device provided in the plurality of processing rooms to perform processing corresponding to a process on a container storing a culture; container moving device for moving the container from a processing room for an upstream process to a processing room for a downstream process adjacent to each other; and control device for controlling the processing device and the container moving device,
the culture processing device moving the container from the processing room for the upstream process to the processing room for the downstream process to perform a series of processing,
the culture processing device further comprising air conditioning device for supplying cleaned gas from upper parts of the processing rooms and discharging the cleaned gas from lower parts of the processing rooms to perform ventilation, characterized in that
when a container storing a culture different from a culture stored in a precedingly processed container is processed,
the container moving device moves the preceding container from the processing room for the upstream process to the processing room for the downstream process adjacent to each other and, thereafter, the air conditioning device ventilates the processing room for the upstream process for a predetermined time and, then, the container moving device carries the container storing the different culture into the processing room for the upstream process.

According to the present invention, when the container storing the different culture is processed, after the preceding container is processed in the processing room, the processing room is ventilated for the predetermined time by the air conditioning device, and then, the container storing the different culture is carried into the processing room.

Accordingly, since splashes and the like caused by the processing performed on the preceding container are eliminated from the processing room by the ventilation, even if the container storing the different culture is carried into the processing room, it is possible to prevent contamination.

### BRIEF DESCRIPTION OF THE DRAWINGS

FIG. 1 is a plan view of a culture processing system according to an embodiment;

FIG. 2 is a side sectional view of a culture device;

FIG. 3 is a front sectional view of a culture processing device; and

FIG. 4 is a diagram illustrating processing in the culture processing device in time series.

### DETAILED DESCRIPTION OF THE PREFERRED EMBODIMENT

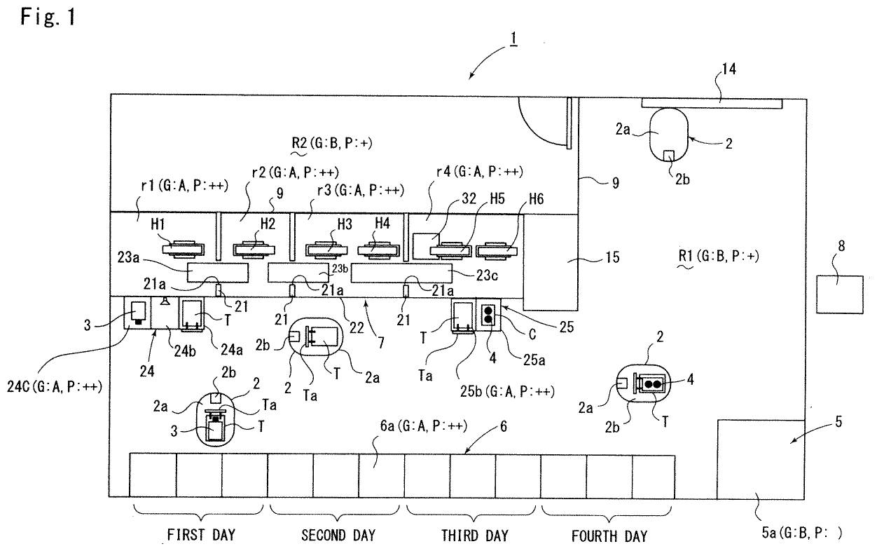

An embodiment illustrated in the figures is explained below. FIG. 1 illustrates a culture processing system 1 that performs processing for a culture. The culture processing system 1 is provided in a culture facility. A treatment room in which a cell is collected from a subject is provided above (or below) a floor where the culture processing system 1 is provided.

The culture processing system 1 in the present embodiment cultures a collected cell for a predetermined period to cause the cell to proliferate and, after the culture, separates a culture into the cell and a supernatant and collects the cell and the supernatant. Note that, in the following explanation, a cell and a culture are sometimes synonymous and indicates the same thing.

The culture processing system 1 includes an unmanned workroom R1 that an operator is prohibited from entering during culture of a cell and a manned workroom R2 in which the operator performs work. In the unmanned workroom R1, a plurality of unmanned conveyors 2 convey containers such as a culture container 3 and a centrifuge tube 4.

In the unmanned workroom R1, a lifting device 5 that delivers a cell to and from the treatment room is provided and a culture device 6 including an incubator that cultures the cell for a predetermined period and a culture processing device 7 according to the present invention that performs processing on a culture are installed. The culture processing system 1 is controlled by control device 8.

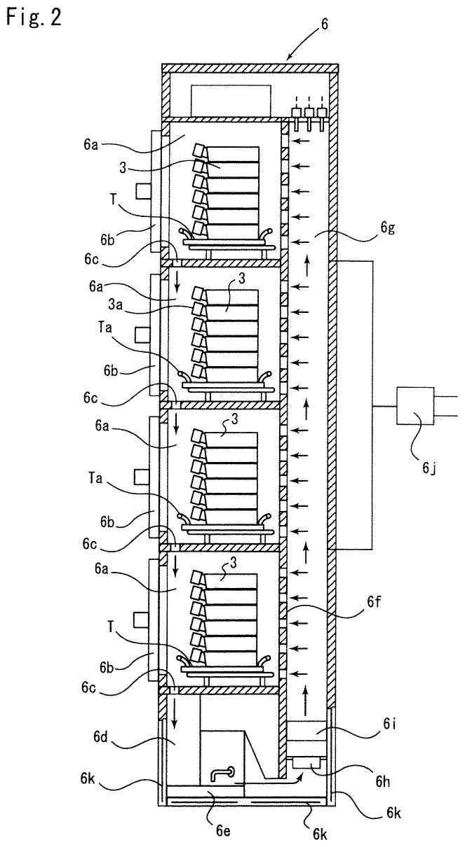

The culture container 3 has a configuration in which a lid 3a is attached to an end portion of a flat substantially rectangular parallelepiped container as illustrated in FIG. 2.

In the present embodiment, in a state in which six culture containers 3 are vertically stacked, the culture containers 3 are housed in a culture room 6a of the culture device 6 to culture cells and are conveyed by the unmanned conveyors 2 in the unmanned workroom R1. The six culture containers 3 are set as one rod as culture containers for one subject.

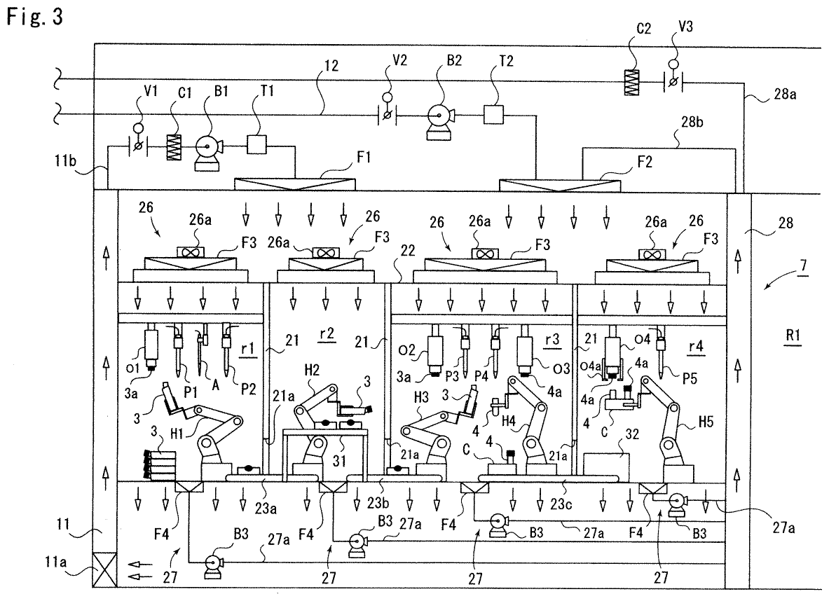

The centrifuge tube 4 has a configuration that can be sealed by attaching a lid 4a thereto. The centrifuge tube 4 is housed in a holding table C capable of holding the centrifuge tube 4 as illustrated in FIG. 3 and conveyed by the unmanned conveyors 2 in the unmanned workroom R1.

Not-illustrated filters that allow only gas to pass are provided in the lid 3a of the culture container 3 and the lid 4a of the centrifuge tube 4. It is possible to cause ambient gas to flow in while preventing cross-contamination.

The unmanned workroom R1 and the manned workroom R2 are configured to be sealed by a wall 9 (illustrated in FIG. 1) provided from the floor to the ceiling such that air cannot flow between the unmanned workroom R1 and the manned workroom R2.

The unmanned workroom R1 and the manned workroom R2 are maintained in an environment having required cleanliness respectively by separate air conditioning device.

Note that, in FIG. 1, signs indicating the unmanned workroom R1 and the manned workroom R2 and first to fourth processing rooms r1 to r4 formed on the inside of the culture processing device 7 explained below are added with indications of cleanliness and pressure on the insides thereof.

The cleanliness is indicated as grade A equivalent (G: A) and grade B equivalent (G: B) in the descending order of the cleanliness according to air cleanliness levels in “the guideline concerning manufacturing of sterile medical products by a sterile technique”. The pressure on the inside is indicated as (P: ++), (P: +), and (P: ) in order from a section set to the highest positive pressure. Note that it is assumed that the section of (P: ) is set to a slightly higher positive pressure than the atmospheric pressure or to the atmospheric pressure.

Both of the insides of the unmanned workroom R1 and the manned workroom R2 are maintained at the grade B equivalent at the cleanliness level of the air and are set to a positive pressure (P: +) with respect to the atmospheric pressure.

The temperature on the inside of the unmanned workroom R1 is set in a range of 32 to 37° C. with respect to 37° C. set as temperature for culturing a cell in the culture device 6 and, in the present embodiment, is set to 32° C.

This temperature is to ease, when the culture container 3 is taken out from the culture room 6a of the culture device 6, heat shock stress that the cell receives because the ambient temperature suddenly drops and to avoid an adverse effect on the quality of the cell due to the heat shock stress. In contrast, the temperature of the manned workroom R2 is set to approximately 25° C. or lower and is set to temperature comfortable for the operator to maintain the manned workroom R2 in an environment suitable for work.

As illustrated in FIG. 3, air conditioning device 10 is configured to perform air conditioning for the unmanned workroom R1 and the culture processing device 7. Here, a portion that performs the air conditioning for the unmanned workroom R1 is explained.

Two sets of air conditioning device for the unmanned workroom R1 are provided. First air conditioning device comprises a filter F1 including a high-performance cleaning filter such as an HEPA or ULPA filter provided on the ceiling of the unmanned workroom R1 and a circulation duct 11 including a circulation port 11a in a lower part of the unmanned workroom R1. The first air conditioning device includes an air blow pipe 11b provided between the filter F1 and the circulation duct 11, an on-off valve V1 provided in the air blow pipe 11b and capable of adjusting an opening degree, a catalyst C1 that absorbs contaminated gas when the inside of the unmanned workroom R1 is decontaminated, a blower B1 that blows air from the circulation duct 11 toward the filter F1, and temperature adjusting device T1 for adjusting the temperature of gas flowing in the air blow pipe 11b.

The gas blown by the blower B1 is adjusted when the gas passes through the temperature adjusting device T1. The gas is supplied from an upper part of the unmanned workroom R1 after being cleaned by the filter F1.

The gas falling to a lower part of the unmanned workroom R1 flows in from the circulation port 11a of the circulation duct 11 and flows in the air blow pipe 11b to be circulated from the filter F1 to the unmanned workroom R1.

Second air conditioning device of the unmanned workroom R1 comprises, on the ceiling of the unmanned workroom R1, a filter F2 including a high-performance cleaning filter such as an HEPA or ULPA filter and an air supply pipe 12 drawn in from the outside of the unmanned workroom R1 and connected to the filter F2. The second air conditioning device includes, in the air supply pipe 12, an on-off valve V2 capable of adjusting an opening degree, a blower B2 that blows air from the outside toward the filter F2, and temperature adjusting device T2 for adjusting the temperature of gas flowing in the air supply pipe 12.

The temperature of gas blown by the blower B2 is adjusted when the gas passes through the temperature adjusting device T2. The gas is supplied from an upper part of the unmanned workroom R1 after being cleaned by the filter F2.

Air gas falling to a lower part of the unmanned workroom R1 flows in from the circulation port 11a of the circulation duct 11 constituting the first air conditioning device and flows in the air blow pipe 11b to be circulated from the filter F1 to the unmanned workroom R1. Here, a plurality of circulation ports 11a are dispersed and formed on the floor surface of the unmanned workroom R1 as well besides the lower part of the circulation duct 11. These circulation ports 11a are also caused to communicate with the circulation duct 11.

Here, the control device 8 adjusts and controls air blow amounts of the blowers B1 and B2, opening and closing amounts of the on-off valves V1 and V2, and the temperature adjusting device T1 and T2 to adjust a circulation amount of gas, an air supply amount of external air, and the temperatures of the gas and the external air in the unmanned workroom R1.

The unmanned conveyors 2 illustrated in FIG. 1 adopt an AIV (an automatic conveyance mobile robot) and have a configuration including a robot hand 2b in an upper part of a carriage 2a that creates a map by itself and is movably provided. The unmanned conveyors 2 is controlled by the control device 8 via radio.

The carriage 2a conveys the culture container 3 and the centrifuge tube 4 in a state of being placed on a tray T. The tray T includes a handle Ta. The robot hand 2b grips the handle Ta and places the tray T on the carriage 2a to be capable of moving the holding table C that holds the culture container 3 and the centrifuge tube 4 together with the tray T.

A charging station 14 is provided in the unmanned workroom R1. The control device 8 automatically performs charging in the charging station 14 while monitoring the remaining battery life.

The lifting device 5 is configured to raise and lower, in the air conditioning duct 5a, a carriage to which gas cleaned by the HEPA or ULPA filter or the like is supplied and move, between a floor where the culture processing system 1 is provided and a floor where the treatment rooms are provided, the culture container 3 and the centrifuge tube 4 placed on the tray T while maintaining a clean state.

The inside of the air conditioning duct 5a is partitioned by the unmanned workroom R1 and is set to the cleanliness of the grade (B) and the (P: ). The inside of the air conditioning duct 5a has a lower pressure than the unmanned workroom R1.

Accordingly, inflow of the atmosphere from the lifting device 5 into the unmanned workroom R1 is suppressed as much as possible.

As illustrated in FIG. 2, the culture device 6 comprises an incubator including a plurality of culture rooms 6a described in Japanese Laid-Open Patent Application No. 2022-184127.

The insides of the culture rooms 6a are maintained at the grade A equivalent at the cleanliness level of the air and are set to the higher positive pressure (P: ++) than the unmanned workroom R1.

In the culture device 6, the culture rooms 6a in four stages in the up-down direction are formed in three rows in the lateral direction. The culture device 6 includes twelve culture rooms 6a in total to constitute one incubator. In the present embodiment, four incubators are coupled in the lateral direction and forty-eight culture rooms 6a in total are included in the culture device 6.

In the culture rooms 6a, opening and closing doors 6b are respectively provided in front loading and unloading entrances.

Upper and lower culture rooms 6a communicate through an exhaust port 6c, a gas merging room 6d is provided below the culture room 6a at the bottom stage, and a humidifying section 6e that generates stream is disposed.

Behind the culture rooms 6a, a gas pressurizing section 6g is formed via a ventilation wall 6f such as punching metal in which a large number of openings are formed. A fan unit 6h that supplies air toward the gas pressurizing section 6g and a high-performance cleaning filter 6i such as an HEPA or ULPA filter are provided between the gas merging room 6d and the gas pressurizing section 6g.

One incubator includes, with respect to one gas merging room 6d and one gas pressurizing section 6g, three sets of fan units 6h and cleaning filters 6i to correspond to the number of rows of the culture rooms 6a.

A culture gas supply source that supplies a culture gas such as a carbon dioxide gas (carbon dioxide) or a nitrogen gas to the culture rooms 6a at the time of culture and a decontamination medium supply source that supplies a decontamination medium such as hydrogen peroxide vapor or hydrogen peroxide mist to the culture rooms 6a at the time of decontamination can be selectively connected to the gas pressurizing section 6g via gas switching device 6j.

At the time of culture, for each one incubator, a gas supply amount of the culture gas supply source is adjusted to maintain the pressure in the culture rooms 6a at a predetermined positive pressure. Further, according to operation of the fan unit 6h, the culture gas is circulated from the culture rooms 6a to the gas merging room 6 d and the gas pressurizing section 6g, the humidity in the culture rooms 6a is maintained at humidity suitable for the culture by the humidifying section 6e, and the insides of the culture rooms 6a are maintained at necessary cleanliness by the cleaning filter 6i.

Heaters 6k are embedded in housings of the incubators to, for each one incubator, maintain the temperature in the culture rooms 6a at temperature suitable for the culture. Further, at the time of the decontamination as well, it is possible to supply the decontamination medium to each one incubator and decontaminate the insides of the culture rooms 6a.

When the culture container 3 is housed in the culture room 6a from the unmanned conveyor 2, the opening and closing door 6b designated by the control device 8 is automatically opened and the culture container 3 stacked and placed on the tray T on a carriage 21b of the unmanned conveyor 2 is housed in the culture room 6a together with the tray T by gripping the hand Ta of the tray T with the robot hand 2b.

When the culture container 3 is taken out from the culture room 6a, the opening and closing door 6b designated by the control device 8 is automatically opened and, as at the time of the housing, the robot hand 2b grips the handle Ta of the tray T and takes out the culture container 3 from the culture room 6a together with the tray T and places the culture container 3 on the carriage 21b.

The culture device 6 in the present embodiment comprises four incubators and includes forty-eight culture rooms 6a in total. Six culture containers 3 are housed in each one culture room 6a and the culture container 3 in which a cell collected from a different subject is planted is housed in each of the culture rooms 6a.

In the present embodiment, the culture containers 3 to be processed on the same day are housed in twelve culture rooms 6a for one incubator. Specifically, six culture containers 3 are housed in each of the culture rooms 6a of a first incubator constituting first to third rows on a first day, six culture containers 3 are housed in each of the culture rooms 6a of a second incubator constituting fourth to sixth rows on a second day, and six culture containers 3 are housed in each of the culture rooms 6a of a third incubator constituting seventh to ninth rows on a third day.

In a fourth day, the culture containers 3 are housed in the culture rooms 6a of a fourth incubator constituting tenth to twelfth rows and all the culture containers 3 in which the culture for the three days has ended are sequentially taken out from the culture rooms 6a of the first incubator constituting the first to third rows.

At the same time, on the fourth day, the gas switching device 6j of the first incubator is switched, a decontamination gas is supplied to the culture rooms 6a in the first to third rows from which the culture containers 3 have been taken out and the decontamination is performed. On a fifth day, new culture containers 3 are housed in the culture rooms 6a of the first incubator.

As explained above, in the present embodiment, seventy-two culture containers 3 in total are processed in twelve rods (for twelve subjects) per day while the culture for the three days being repeated. Note that the number of culture containers 3 to be processed is not limited to this. It is also possible to process a larger number of culture containers 3 in one day by increasing the number of culture rooms 6a.

As illustrated in FIG. 1, an observation device 15 for checking a state of a culture being cultured is provided at a position adjacent to the culture processing device 7 in the unmanned workroom R1.

A microscope or the like for observing a culture in the culture container 3 is provided in the observation device 15.

When the unmanned conveyor 2 places the culture container 3 at a predetermined observation position of the observation device 15, the operator checks an image of the microscope and performs observation from the manned workroom R2 side.

FIG. 3 illustrates the culture processing device 7. The culture processing device 7 includes a housing 22, the inside of which is partitioned into first to fourth processing rooms r1 to r4 associated with processing processes by partition walls 21 with communication ports 21a below, processing device for performing processing corresponding to the processes on the culture containers 3 and the centrifuge tubes 4 provided in the first to fourth processing rooms r1 to r4, and container moving device 23 (23a, 23b, and 23c) for moving culture containers 3 and the centrifuge tubes 4 from a processing room r for an upstream process to the processing room r for a downstream process adjacent to each other. The culture processing device 7 moves the culture containers 3 and the centrifuge tubes 4 to the first to fourth processing rooms r1 to r4 and performs a series of processing.

The housing 22 is provided on the inside of the unmanned workroom R1 as illustrated in FIG. 1. As illustrated in FIG. 3, the ceiling of the housing 22 is located below the ceiling of the unmanned workroom R1 and the floor surface of the housing 22 is located above the floor surface of the unmanned workroom R1.

The insides of the first to fourth processing rooms r1 to r4 of the culture processing device 7 are maintained at the grade A equivalent at the cleanliness level of the air by the air conditioning device 10, maintained at a positive pressure (P: ++) higher than the pressure in the unmanned workroom R1 in the periphery, and the temperature on the insides is maintained at approximately 32° C. as in the unmanned workroom R1.

Specifically, in the housing 22, air supply sections 26 that are provided to correspond to the processing rooms r and clean and supply gas in the unmanned workroom R1 and exhaust sections 27 that are provided to correspond to the processing rooms r and discharge gas from the processing rooms r are provided. The gas discharged by the exhaust sections 27 are discharged to the outside by an exhaust duct 28.

The air supply sections 26 comprise filters F3 including high-performance cleaning filters such as HEPA or ULPA filters provided on the respective ceilings that are upper parts of the first to fourth processing rooms r1 to r4 of the housing 22 and fans 26a for supplying air to the filters F3.

The fans 26a supply gas having cleanliness of the grade B equivalent in the unmanned workroom R1 to the filters F3 and clean the gas with the filters F3 to thereby supply the gas to each of the first to fourth processing rooms r1 to r4 as gas having cleanliness of the grade A equivalent.

At this time, since the gas in the unmanned workroom R1 is supplied to the first to fourth processing rooms r1 to r4, the temperature in the insides of the first to fourth processing rooms r1 to r4 is also maintained at approximately 32° C. adjusted by the temperature adjusting device T1 and T2.

The exhaust sections 27 include filters F4 including high-performance cleaning filters such as HEPA or ULPA filters provided on the floor surfaces that are lower parts of the first to fourth processing rooms r1 to r4 and prepared for a case in which outdoor air flows back, collection pipes 27a connected to the filter F4, and blowers B3 provided in the collection pipes 27a. The gas flowing in the first to fourth processing rooms r1 to r4 is sucked by the filters F4 and then discharged to the exhaust duct 28.

A lower part of a sidewall of the housing 22 is opened while having a gap between the lower part and the floor surfaces of the first to fourth processing rooms r1 to r4. The gas in the first to fourth processing rooms r1 to r4 flows out to the unmanned workroom R1. The flowed-out gas is discharged from the circulation port 11a in the lower part of the circulation duct 11.

The exhaust duct 28 includes an exhaust pipe 28a opened to the outside and a branch pipe 28b connected to the filter F2 constituting the second air conditioning device of the unmanned workroom R1 and comprises an on-off valve V3 provided in the exhaust pipe 28a and a catalyst C2 that absorbs a decontamination gas.

Gas discharged by the exhaust section 27 from the first to fourth processing rooms r1 to r4 flows in the collection pipe 27a to be gathered by the exhaust duct 28 and is, thereafter, discharged to the outside according to an opening amount of the on-off valve V3 provided in the exhaust pipe 28a, and the gas not discharged is cleaned by the filter F2 via the branch pipe 28b and circulated to the unmanned workroom R1.

The control device 8 adjusts a flow rate of gas supplied to the processing rooms r by the air supply sections 26, an air volume of the blowers B3 of the exhaust sections 27, and an opening amount of the on-off valve V3 of the exhaust pipe 28a and maintains the insides of the processing rooms r at a positive pressure (P: ++).

The air supply sections 26 supply air from the upper parts of the processing rooms r and the exhaust sections 27 exhaust and ventilate the processing rooms r from the lower parts thereof and gas is always discharged into the unmanned workroom R1 from side lower parts of the processing rooms r, whereby laminar flows flowing downward from the upper parts are formed on the insides of the first to fourth processing rooms r1 to r4. Here, the air supply sections 26 and the exhaust sections 27 are caused to always operate during processing for a culture.

Even in a state in which processing for the culture containers 3 and the centrifuge tubes 4 is not performed in the first to fourth processing rooms r1 to r4, the ventilation of the processing rooms r is continued.

The container moving device 23 includes first to third conveyors 23a to 23c provided between the processing rooms r adjacent to each other, carry-in device for carrying the culture container 3 into the first processing room r1 from a carry-in section 24, and carry-out device for carrying out the centrifuge tube 4 to a carry-out section 25 from the fourth processing room r4.

The first to third conveyors 23a to 23c are provided to pass through communication ports 21a formed in a partition wall 21.

The first conveyor 23a is provided to convey the culture container 3 from the first processing room r1 for an upstream process to the second processing room r2 for a downstream process. The second conveyor 23b is provided to convey the culture container 3 from the second processing room r2 for an upstream process to the third processing room r3 for a downstream process. The third conveyor 23c is provided to convey the centrifuge tube 4 from the third processing room r3 for an upstream process to the fourth processing room r4 for a downstream process.

Here, the communication ports 21a are configured to cause the processing rooms r adjacent to each other to always communicate. In other words, the communication ports 21a do not have a configuration opened and closed by an opening and closing door. The communication ports 21a are capable of moving the culture container 3 and the centrifuge tube 4 to the adjacent processing room r with the conveyors 23a to 23c without involving an opening and closing operation.

As illustrated in FIG. 1, the carry-in section 24 includes a tray placing section 24a on which the culture container 3 is placed together with the tray T, an alcohol spray section 24b that sprays alcohol to the culture container 3, and a drying section 24c that dries the alcohol.

In the carry-in section 24, a not-illustrated robot hand or conveyor conveys the culture container 3. When stacked six culture containers 3 are placed on the tray placing section 24a, the culture containers 3 are conveyed to the alcohol spray section 24b and the drying section 24c one by one.

The alcohol spray section 24b and the drying section 24c are covered by a required housing. The drying section 24c and the first processing room r1 communicate via a communication port.

The culture containers 3 are stacked again in the drying section 24c, held by carry-in device such as a not-illustrated robot hand or conveyor, and carried into the first processing room r1. The inside of the drying section 24c is set to cleanliness of the grade A and a high positive pressure (P: ++) by communicating with the first processing room r1. The alcohol spray section 24b and the tray placing section 24a are maintained in an environment equivalent to the unmanned workroom R1.

The carry-out section 25 includes a carry-out room 25a to which the culture container 3 is discharged from the fourth processing room r4 and a tray placing section 25b on which the tray T is placed. Two centrifuge tubes 4 held by the holding table C in the fourth processing room r4 are carried out to the carry-out room 25a by carry-out device such as a not-illustrated robot hand or conveyor.

The carry-out room 25a is covered by a required housing and communicates with the fourth processing room r4 via a communication port. The inside of the carry-out room 25a is set to the cleanliness grade A and a high positive pressure (pressure ++).

The tray T emptied in the tray placing section 24a of the carry-in section 24 is conveyed to and placed on the tray placing section 25b by the unmanned conveyor 2. The holding table C holding the two centrifuge tubes 4 in the carry-out room 25a is placed on the tray T. The tray placing section 25b is maintained in an environment equivalent to the unmanned workroom R1.

On the inside of the first processing room r1, a first processing process of removing culture liquid in the culture container 3 and supplying a peeling agent is performed.

Processing device of the first processing room r1 includes first opening and closing device O1 for attaching and detaching the lid 3a to and from the culture container 3, a first pipette P1 that supplies cleaning liquid to the culture container 3, an aspirator A that discharges the culture liquid and the cleaning liquid from the culture container 3, and a second pipette P2 that supplies the peeling agent to the culture container 3.

These are provided above the communication port 21a of the partition wall 21.

As the processing device, in the first processing room r1, a first robot hand H1 serving as holding and moving device for holding the culture container 3 and moving the culture container 3 upward and downward is provided. The six culture containers 3 supplied from the drying section 24c of the carry-in section 24 can be held one by one and moved above the communication port 21a.

Note that the six culture containers 3 stacked on the drying section 24c may be held and carried in one by one using the first robot hand H1 as carry-in device.

When holding the culture containers 3 one by one, the first robot hand H1 sequentially moves the culture container 3 to the first opening and closing device O1, the first pipette P1, the aspirator A, and the second pipette P2 and moves the culture container 3 to the first opening and closing device O1 again.

The first opening and closing device O1 grips and rotates to detach the lid 3a attached to the culture container 3 held by the first robot hand H1 and, when the culture container 3 is moved to the first opening and closing device O1, rotates the gripped lid 3a reversely to the preceding rotation and attaches the lid 3a to the culture container 3 again.

The first pipette Pl sucks and ejects liquid. The first pipette P1 sucks a predetermined amount of cleaning liquid from a cleaning liquid bottle raised by a not-illustrated lifting mechanism provided in the first processing room r1 and ejects the cleaning liquid into the culture container 3.

The aspirator A sucks the culture liquid and the cleaning liquid in the culture container 3 and discharges the culture liquid and the cleaning liquid to a not-illustrated waste liquid tank.

The second pipette P2 sucks and ejects liquid. The second pipette P2 sucks a predetermined amount of a peeling agent from a peeling agent bottle lifted by the not-illustrated lifting mechanism provided in the first processing room r1 and ejects the peeling agent into the culture container 3.

When the lid 3a is attached to the culture container 3, from which the culture liquid is removed and to which the peeling agent is supplied, again in the first opening and closing device O1, after being moved by the first robot hand H1 to height where the culture container 3 can pass through the communication port 21a of the partition wall 21 below, the culture container 3 is placed on the first conveyor 23a, conveyed, and carried into the second processing room r2.

On the inside of the second processing room r2, a second processing process of heating and peeling a cell in the culture container 3 is performed.

Plate-like heating device 31 is provided as processing device of the second processing room r2. The heating device 31 is provided above the communication port 21a of the partition wall 21.

As the processing device, in the second processing room r2, a second robot hand H2 serving as holding and moving device for holding the culture container 3 and moving the culture container 3 upward and downward is provided. The second robot hand H2 holds the culture containers 3, which are supplied from the first processing room r1 to the second processing room r2 by being conveyed by the first conveyor 23a, one by one and moves the culture container 3 to the heating device 31 above the communication port 21a and places the culture container 3 on the heating device 31.

A cell in the culture container 3 is peeled from the culture container 3 by heating the culture container 3 with the heating device 31.

After being held by the second robot hand H2 and moved to height where the culture container 3 can pass through the communication port 21a of the partition wall 21 below, the heated culture container 3 is placed on the second conveyor 23b, conveyed, and carried into the third processing room r3.

On the inside of the third processing room r3, a third processing process of collecting a cell from the culture container 3 is performed.

Processing device of the third processing room r3 includes second opening and closing device O2 for attaching and detaching the lid 3a to and from the culture container 3, a third pipette P3 that supplies a neutralizing medium to the culture container 3, a fourth pipette P4 that sucks the neutralizing medium and the cell in the culture container 3 and ejects the neutralizing medium and the cell to the centrifuge tube 4, and third opening and closing device O3 for attaching and detaching the lid 4a to and from the centrifuge tube 4. These are provided above the communication port 21a of the partition wall 21.

As the processing device, in the third processing room r3, a third robot hand H3 serving as holding and moving device for holding the culture container 3 and moving the culture container 3 upward and downward and a fourth robot hand H4 serving as holding and moving device for holding the centrifuge tube 4 supplied to the third processing room r3 as a material in advance and moving the centrifuge tube 4 upward and downward are provided.

Here, two centrifuge tubes 4 including the centrifuge tube 4 used in the next process are held by the holding table C and supplied. The fourth robot hand H4 holds, in advance, the holding table C holding the two centrifuge tubes 4 and places the holding table C on the third conveyor 23c.

When holding the culture containers 3 one by one from the second conveyor 23b, the third robot hand H3 sequentially moves the culture container 3 to the second opening and closing device 02, the third pipette P3, and the fourth pipette P4 and moves the culture container 3 to the second opening and closing device O2 again.

The second opening and closing device O2 grips and rotates to detach the lid 3a attached to the culture container 3 held by the third robot hand H3 and, when the culture container 3 is moved to the second opening and closing device O2 again, rotates the gripped lid 3a reversely to the preceding rotation and attaches the lid 3a to the culture container 3 again. The culture container 3 to which the lid 3a is attached again is collected in a not-illustrated collection box.

The third pipette P3 sucks a predetermined amount of a neutralizing medium from a neutralizing medium bottle raised by a not-illustrated lifting mechanism provided in the third processing room r3 and ejects the neutralizing medium into the culture container 3.

When holding one centrifuge tube 4 on the holding stand C placed on the third conveyor 23c, the fourth robot hand H4 sequentially moves the centrifuge tube 4 to the third opening and closing device O3 and the fourth pipette P4 and moves the centrifuge tube 4 to the third opening and closing device O3 again.

When sucking a cell together with the neutralizing medium from the culture container 3 held by the third robot hand H3, the fourth pipette P4 ejects the cell and the neutralizing medium to the centrifuge tube 4 held by the fourth robot hand H4.

The third opening and closing device O3 grips and rotates to detach the lid 4a attached to the centrifuge tube 4 held by the fourth robot hand H4 and, when the centrifuge tube 4 is moved to the third opening and closing device O3 again, rotates the gripped lid 4a reversely to the preceding rotation and attaches the lid 4a to the centrifuge tube 4 again.

The third robot hand H3 moves the emptied culture container 3 downward after attaching the lid 3a with the second opening and closing device O2 and discards the culture container 3 in a not-illustrated disposal box.

In the third processing process in the third processing room r3, an operation of supplying the neutralizing medium to the culture container 3 with the third pipette P3 and sucking the cell together with the neutralizing medium from the culture container 3 with the fourth pipette P4 and ejecting the cell and the neutralizing medium to the centrifuge tube 4 is repeated six times and one centrifuge tube 4 is caused to collectively store a suspension including the neutralizing medium and the cell sucked from the six culture containers 3.

The fourth robot hand H4 moves the centrifuge tube 4 storing the suspension to height where the centrifuge tube 4 can pass through the communication port 21a of the partition wall 21 below after attaching the lid 4a to the centrifuge tube 4 with the third opening and closing device O3 and causes the holding stand C placed on the third conveyor 23c to hold the centrifuge tube 4.

When the centrifuge tube 4 is held by the holding stand C, in a state in which the centrifuge tube 4 is held by the holding stand C, the third conveyor 23c conveys the centrifuge tube 4 storing the suspension and the empty centrifuge tube 4 from the third processing room r3 to the fourth processing room r4 and carries the centrifuge tubes 4 into the fourth processing room r4.

In the fourth processing room r4, a fourth processing process of separating the suspension into the cell and a supernatant is performed. In the present embodiment, the fourth processing process is a final process and the fourth processing room r4 is a processing room of the final process.

Processing device of the fourth processing room r4 includes centrifugal separation device 32 for centrifugally separating a culture in the centrifuge tube 4, fourth opening and closing device O4 for attaching and detaching the lid 4a to and from the centrifuge tube 4 in the state of being held by the holding table C, and a fifth pipette P5 that sucks the supernatant from the centrifuge tube 4 storing the suspension and transfers the supernatant to the empty centrifuge tube 4. These are provided above the communication port 21a of the partition wall 21.

As the processing device, in the fourth processing room r4, a fifth robot hand H5 serving as holding and moving device for directly holding the centrifuge tube 4 or holding the holding table C holding the two centrifuge tubes 4 and moving the two centrifuge tubes 4 upward and downward is provided.

When the two centrifuge tubes 4 are moved from the third processing room r3 to the fourth processing room r4 in the state of being held by the holding table C, the fifth robot hand H5 takes out the centrifuge tube 4 storing the suspension from the holding table C and moves the centrifuge tube 4 to the centrifugal separation device 32.

The centrifugal separation device 32 centrifugally separates the suspension in the centrifuge tube 4 for a predetermined time. Accordingly, the suspension is separated into the cell and the supernatant.

Subsequently, when taking out the centrifuge tube 4 from the centrifugal separation device 32, the fifth robot hand H5 returns the centrifuge tube 4 to the holding table C, holds the holding table C, sequentially moves the centrifuge tube 4 to the fourth opening and closing device O4 and the fifth pipette P5, and moves the centrifuge tube 4 to the fourth opening and closing device O4 again.

The fourth opening and closing device O4 rotates to detach the lids 4a attached to the two centrifuge tubes 4 held by the holding table C and, when the centrifuge tube 4 is moved to the fourth opening and closing device O4 again, rotates the lids 4a reversely to the preceding rotation and attaches the lids 4a to the culture container 3.

In this case, when gripping to detach the lid 4a of one centrifuge tube 4, the fourth opening and closing device O4 temporarily places the lid 4a on a not-illustrated temporary stand moved downward and, subsequently, grips to detach the lid 4a of the other centrifuge tube 4.

When attaching the lid 4a again, after attaching the gripped lid 4a to the other centrifuge tube 4, the fourth opening and closing device O4 grips the lid 4a on the not-illustrated temporary stand moved downward and attaches the lid 4a to the one centrifuge tube 4.

Here, the fourth opening and closing device O4 includes a gripper O4a that grips the centrifuge tube 4 held by the holding table C not to turn together the lid 4a.

The fifth pipette P5 sucks only the supernatant from the centrifuge tube 4 held by the holding stand C held by the fifth robot hand H5, subsequently, laterally moves the holding table C held by the fifth robot hand H5, and ejects the sucked Supernatant to the other empty centrifuge tube 4.

The fourth opening and closing device O4 sequentially attaches the lids 4a to the two centrifuge tubes 4 held by the fifth robot hand H5 moving the holding stand C.

The fifth robot hand H5 moves the holding table C holding the two centrifuge tubes 4, to which the lids 4a are attached, to the carry-out room 25a of the carry-out section 25.

The holding table C and the centrifuge tubes 4 placed in the carry-out room 25a are placed, by a not-illustrated robot hand or conveyor, on the tray T placed on the tray placing section 25b and are taken out from the carry-out section 25 with the robot hand 2b of the unmanned conveyor 2 together with the tray T.

Note that, as carry-out device for carrying out the centrifuge tubes 4 from the fourth processing room r4 to the carry-out section 25, a not-illustrated robot hand or conveyor can also be separately provided besides the fifth robot hand H5.

In the culture processing device 7 in the present embodiment, the processing by the processing device such as the pipette P, the aspirator A, and the opening and closing device O is performed above the communication port 21a formed in the partition wall 21 that partitions the processing rooms r.

In other words, the robot hands H move the culture containers 3 and the centrifuge tubes 4 to above the communication port 21a, open the culture containers 3 and the centrifuge tubes 4 in an upper position, and cause the processing device to perform the processing.

In the present embodiment, since the processing room r for the upstream process and the processing room r for the downstream process adjacent to each other are caused to always communicate by the communication port 21a, even if a laminar flow (a one-way flow) is formed from an upper part to a lower part of the processing room r, atmosphere including splashes caused from a culture is likely to flow into the adjacent processing room r.

However, in the present embodiment, since the processing explained above performed by opening the culture container 3 and the centrifuge tube 4 is performed above the communication port 21a, even if the atmosphere including the splashes flows into the adjacent processing room r from the communication port 21a, the atmosphere is prevented from flowing upward from the communication port 21a by the laminar flow flowing downward. It is possible to prevent, as much as possible, the atmosphere from mixing in the culture container 3 and the centrifuge tube 4 opened above the communication port 21a.

On a side surface facing the manned workroom R2 in the culture processing device 7, a not-illustrated pass box and not-illustrated gloves are provided in order to supply materials and the like necessary for the processing in the processing rooms r.

As the materials and the like, besides the pipette and the centrifuge tube, there is, for example, a bottle storing liquid such as cleaning liquid or a peeling agent.

That is, in the manned workroom R2, the operator is capable of carrying, into the pass box, the materials and the like stored in a packaging bag, sterilized by gamma ray sterilization or the like, and supplied, decontaminating the outer surface of the packaging bag with a decontamination medium such as hydrogen peroxide vapor or hydrogen peroxide mist in the pass box, and, thereafter, taking out the materials and the like from the packaging bag in the pass box using the gloves provided in the pass box, opening the pass box to the culture processing device 7, and supplying the sterilized materials and the like to the processing rooms r while maintaining a sterile state.

An operation of the culture processing system 1 having the configuration explained above is explained below.

First, a cell is collected from a subject in a treatment room and is planted dividedly in six culture containers 3. When a cell is collected from a different subject, the cell is planted in other new six culture containers 3 with attention paid to contamination.

Subsequently, in the treatment room, the six culture containers 3 are placed on the tray T in a stacked state. The tray T is moved to the culture processing system 1 illustrated in FIG. 1 by the lifting device 5. The culture containers 3 in which the cell collected from the different subject is planted are placed on another tray T and separately moved.

In the culture processing system 1, the unmanned conveyor 2 stays on standby at a position adjacent to the lifting device 5 in the unmanned workroom R1. The robot hand 2b of the unmanned conveyor 2 holds the tray T, on which the culture containers 3 are placed, and transfers the tray T onto the carriage 2a.

Here, the unmanned workroom R1 is maintained at 32° C. or higher by the air conditioning device 10 and is brought close to temperature at the time when culture is to be performed by the culture device 6.

Subsequently, the unmanned conveyor 2 moves the culture containers 3 to the culture device 6.

The culture device 6 includes forty-eight culture rooms 6a in four stages and twelve rows. In a first day, six culture containers 3 for one person are housed in one of the twelve culture rooms 6a in the first to third rows. Culture of cells of twelve subjects is started in one day.

Note that the number of culture rooms 6a provided in the culture device 6 can be increased or reduced according to the number of subjects who desire culture of cells of the subjects. The insides of the culture rooms 6a are maintained in an environment suitable for culture of cells, for example, at temperature of 37° C., humidity of 95%, and carbon dioxide gas concentration of 5%. When the six culture containers 3 in the state of being placed on the tray T are housed in the culture rooms 6a, thereafter, cells planed in the culture containers 3 are cultured for three days.

In second and subsequent days, each six culture containers 3 are supplied from the treatment room. The culture containers 3 are respectively housed in the culture rooms 6a in the fourth to sixth rows of the culture device 6 in the second day, in the culture rooms 6a in the seventh to ninth rows in the third day, and in the culture rooms 6a in the tenth to twelfth rows in the fourth day.

Culture states of the cells in the culture containers 3 housed in the culture devices 6 are checked by using the observation device 15 in the second day from the culture start.

The culture containers 3 housed in the culture rooms 6a of the culture device 6 are temporarily taken out for each of the trays T and moved to the observation device 15 by the unmanned conveyor 2. The operator checks the culture states from the manned workroom R2.

When the observation by the observation device 15 ends, the unmanned conveyor 2 returns the observed culture containers 3 to the original culture rooms 6a of the culture device 6.

The culture device 6 cultures the cells for three days. On the third day, the unmanned conveyor 2 takes out, for each of the trays T, six culture containers 3 for each of the subjects from the culture rooms 6a in the first to third rows of the culture device 6, moves the six culture containers 3 to the tray placing section 24a of the carry-in section 24 of the culture processing device 7, and places the six culture containers 3 on the tray placing section 24a.

In the culture processing device 7, a series of processing is divided into four processing processes and is sequentially performed in the first to fourth processing rooms r1 to r4. As explained above, the first processing process of removing the culture liquid in the culture containers 3 and supplying the peeling agent is performed in the first processing room r1, the second processing process of heating and peeling the cells in the culture containers 3 is performed in the second processing room r2, the third processing process of collecting the cells from the culture containers 3 is performed in the third processing room r3, and the fourth processing process of separating the suspension into the cells and supernatants is performed in the fourth processing room r4.

Here, in the first to fourth processing rooms r1 to r4, cleaned gas flows in one direction while forming a laminar flow from the ceilings in the upper parts to the floor surfaces in the lower parts. The processing is performed in spaces with high cleanliness in which ventilation is always performed.

The two centrifuge tubes 4 storing the cells and the supernatants obtained as explained above are taken out from the carry-out section 25 of the culture processing device 7 by the unmanned conveyor 2 in a state of being placed on the tray T held by the holding stand C. The unmanned conveyor 2 moves the centrifuge tubes 4 to the lifting device 5 together with the holding table C in a state of being placed on the tray T and the centrifuge tubes 4 are moved to the treatment room by the lifting device 5.

The series of operations explained above is processing for a cell collected from one subject. The processing explained above is continuously performed for the culture container 3 in which a different cell collected from a different subject is planted.

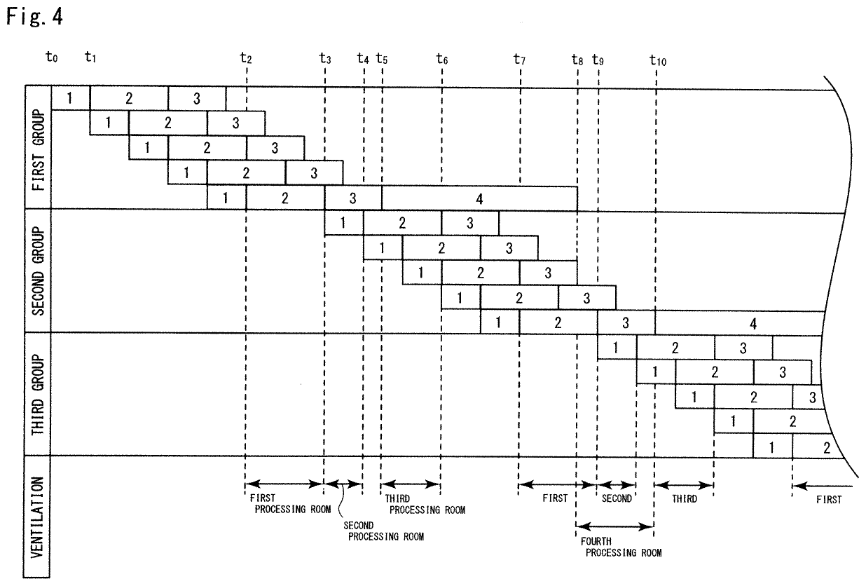

FIG. 4 illustrates, over time, in which processing room r is the culture container 3 from which the cell collected from the different subject is planted is processed.

Here, the cell collected from the one subject is planted dividedly in five culture containers 3. Five culture containers 3 carried into the culture processing device 7 first is explained as a first group, five culture containers 3 following the first group is explained as a second group, and five culture containers 3 following the second group is explained as a third group.

In FIG. 4, the horizontal axis indicates an elapsed time and numbers added to blocks indicate that the culture containers 3 are processed in the first to fourth processing rooms r1 to r4 corresponding to the numbers.

First, when the five culture containers 3 of the first group are carried into the first processing room r1 of the culture processing device 7, processing in the first processing room r1 is sequentially started from a first culture container 3 (time point: t0).

When the processing for the first culture container 3 in the first processing room r1 ends and the first culture container 3 is moved to the second processing room r2 (time point: t1), processing for a second culture container 3 of the first group already housed in the first processing room r1 is started.

Thereafter, processing for third to fifth culture containers 3 is sequentially performed.

The first culture container 3 is processed in the second and third processing rooms r2 and r3, processing for the five culture containers 3 is performed in the third processing room r3, and a suspension for the five culture containers 3 is stored in one centrifuge tube 4.

Thereafter, the centrifuge tube 4 storing the suspension and the empty centrifuge tube 4 are moved from the third processing room r3 to the fourth processing room r4. Processing for separating the suspension into the cell and a supernatant is started in the fourth processing room r4 (time point: t5).

While the processing is sequentially performed on the five culture containers 3 of the first group in the first processing room r1 as explained above, the five culture containers 3 of the second group are prepared in the carry-in section 24 by the unmanned conveyor 2.

However, the culture container 3 of the second group is not carried into the first processing room r1 during the processing for the first group. Thereafter, even if the fifth culture container 3 of the first group is further moved from the first processing room r1 to the second processing room r2 (time point: t2), the culture container 3 of the second group is not carried into the first processing room r1.

In the present embodiment, the five culture containers 3 of the second group are put on standby until all of the five culture containers 3 of the first group are moved from the second processing room r2 to the third processing room r3 (time point: t3), at that time point, the five culture containers 3 of the second group are carried into the first processing room r1.

Accordingly, processing is not performed in the first processing room r1 from when the fifth culture container 3 of the first group is moved to the second processing room r2 (time point: t2) until when the following five culture containers 3 of the second group are carried into the first processing room r1 (time point: t3).

Since the ventilation by the air conditioning device 10 is always performed in the first processing room r1 even in that period, in the processing performed on the culture container 3 of the first group, even if splashes and the like occur from the culture container 3, the splashes or the like are completely eliminated before the processing for the second group is started.

At this time, besides performing exhaust via the collection pipe 27a provided on the floor surface of the first processing room r1, the air conditioning device 10 performs exhaust from an exhaust gap formed in a lower part of the sidewall of the housing 22 to the unmanned workroom R1 on the outside. Therefore, the splashes and the like that have occurred in the first processing room r1 can be discharged as much as possible.

As explained above, the processing for the culture container 3 of the second group in the first processing room r1 is started at timing when the fifth culture container 3 of the first group has moved to the third processing room r3 (time point: t3).

At this time, in the second processing room r2, the first culture container 3 of the first group has moved to the third processing room r3. Therefore, the second processing room r2 is emptied.

Accordingly, the second processing room r2 is maintained in the empty state until the first culture container 3 of the second group is moved from the first processing room r1 to the second processing room r2 (time point: t4). In that period, the second processing room r2 is ventilated by the air conditioning device 10 in the same manner as when the first processing room r1 is emptied.

A culture of the first group is stored in one centrifuge tube 4 in the third processing room r3 and moves to the fourth processing room r4 (time point: t5). Thereafter, the third processing room r3 is emptied until the first culture container 3 of the second group is carried into the third processing room r3 (time point: t6). In that period, the third processing room r3 is ventilated by the air conditioning device 10 in the same manner as when the first processing room r1 is emptied.

Thereafter, processing is respectively performed in the first to fourth processing rooms r1 to r4 until the processing for the fifth culture container 3 of the second group ends in the first processing room r1 (time point: t7).

The processing for the centrifuge tube 4 of the first group ends in the fourth processing room r4 (time point: t8).

Thereafter, the fourth processing room r4 is emptied until the centrifuge tube 4 of the second group moves from the third processing room r3 to the fourth processing room r4 (time point: t10). In that period, the fourth processing room r4 is ventilated by the air conditioning device 10 in the same manner as when the first processing room r1 is emptied.

On the other hand, when processing for the third group is started following the second group in the culture processing device 7 and the culture container 3 of the third group is carried into the first processing room r1 (time point: t9), since the fifth culture container 3 of the second group has moved from the second processing room r2 to the third processing room r3, the first processing room r1 is ventilated in that period.

Thereafter, subsequently, the culture containers 3 of the fourth and fifth groups are supplied to the culture processing device 7. When the culture containers 3 of the different groups are processed in this way, the processing for the following group is performed after the processing rooms r are ventilated.

As explained above, with the culture processing device 7 in the present embodiment, since the processing room r for the upstream process and the processing room r for the downstream process adjacent to each other always communicate through the communication port 21a, containers such as the culture container 3 and the centrifuge tube 4 can be quickly moved. It is possible to perform efficient culture processing.

In the present embodiment, in the case of processing different cells of subjects, when processing for a cell of the preceding subject ends, processing for a cell of the following subject is not immediately performed and the processing room r is ventilated for a predetermined time in an empty state.

That is, when the processing for the cell of the preceding subject is performed, even if splashes and the like deriving from the cell of the subject have occurred, the splashes and the like can be eliminated by the ventilation.

Accordingly, when the processing for the cell of the following different subject is performed in the ventilated processing room r, it is possible to prevent, as much as possible, splashes and the like deriving from the cell of the preceding subject from mixing in the containers such as the culture container 3 and the centrifuge tube 4 and it is possible to prevent contamination.

Note that a ventilation time, that is, a time in which the processing room r is brought into the empty state is set as appropriate based on the size of the processing room r, a time in which the culture container 3, the centrifuge tube 4, and the like are opened, a ventilation air volume, and the like such that the splashes and the like are sufficiently eliminated.

In the culture processing device 7 in the present embodiment, the processing for the containers such as the culture container 3 and the centrifuge tube 4 is performed above the communication port 21a formed in the partition wall 21 that partitions the processing room r for the upstream process and the processing room r for the downstream process adjacent to each other.

Since the laminar flow from the upper part to the lower part is formed in the processing room r by the air conditioning device 10, even if splashes and the like flow into the processing room r from the adjacent processing room r, the splashes and the like are prevented as much as possible from flying above the communication port 21a. Therefore, it is possible to completely prevent contamination as long as the processing is performed above the communication port 21a.

Here, in the embodiment explained above, as illustrated in FIG. 4, the processing for the first culture container 3 of the second group is started in the second processing room r2 at the timing when the processing for the fifth culture container 3 of the first group is started in the third processing room r3 (time point: t4).

That is, from the time point: t4 until the fifth culture container 3 of the first group moves to the fourth processing room r4 (time point: t5), the processing for the cells of the different subjects is performed in the third processing room r3 and the fourth processing room r4 adjacent to each other.

However, in the present embodiment, since processing in which the lid 3a is opened is not performed in the second processing room r2, even if inflow of splashes and the like occurs between the third processing room r3 and the second processing room r2, contamination does not occur.

Note that, in order to avoid such a state, start timing of the processing for the second group only has to be slightly delayed. Specifically, end timing (t4 in FIG. 4) of the processing for the second group in the first processing room r1 only has to be aligned with timing (t5 in FIG. 4) when the processing for the centrifuge tube 4 of the first group in the fourth processing room r4 is started.

Note that the culture processing system 1 in the present embodiment cultures, only for three days, a cell collected from a subject and collects the cell. However, the culture processing system 1 may be configured to perform other processing for a culture.

For example, when the cell is cultured for a longer period, processing such as medium change and subculture for a culture container is necessary. Therefore, processing device, a container, and the like necessary for the medium change can be provided as the processing device in the processing room r of the culture processing device 7. The processing processes are not limited to the four processing processes and can be increased or reduced.

Note that the container moving device 23 for conveying the containers such as the culture container 3 and the centrifuge tube 4 between the adjacent processing rooms r is not limited to the conveyor on which the containers such as the culture container 3 and the centrifuge tube 4 are placed and conveyed and may be handling device for holding and moving the containers such as the culture container 3 and the centrifuge tube 4. The containers such as the culture container 3 and the centrifuge tube 4 can also be handed over between the robot hands H provided in the processing rooms r.

### REFERENCE SIGNS LIST

1 Culture processing system
2 Unmanned conveyor
3 Culture container (Container)
4 Centrifuge tube (Container)
6 Culture device
7 Culture processing device
10 Air conditioning device
21 Partition wall
21a Communication port
23 Container moving device
r1 to r4 First to fourth processing rooms
R1 Unmanned workroom
R2 Manned workroom
A Aspirator (Processing device)
O Opening and closing device (Processing device)
P Pipette (Processing device)
H Robot hand (Holding and moving device)

1. A culture processing device comprising: a plurality of processing rooms partitioned to correspond to a plurality of processing processes; processing device provided in the plurality of processing rooms to perform processing corresponding to a process on a container storing a culture; container moving device for moving the container from a processing room for an upstream process to a processing room for a downstream process adjacent to each other; and control device for controlling the processing device and the container moving device,
the culture processing device moving the container from the processing room for the upstream process to the processing room for the downstream process to perform a series of processing, wherein
the culture processing device further comprising air conditioning device for supplying cleaned gas from upper parts of the processing rooms and discharging the cleaned gas from lower parts of the processing rooms to perform ventilation,
when a container storing a culture different from a culture stored in a precedingly processed container is processed,
the container moving device moves the preceding container from the processing room for the upstream process to the processing room for the downstream process adjacent to each other and, thereafter, the air conditioning device ventilates the processing room for the upstream process for a predetermined time and, then, the container moving device carries the container storing the different culture into the processing room for the upstream process.

2. The culture processing device according to claim 1, wherein
when the container storing the culture different from the culture stored in the precedingly processed container is processed in a processing room for a final process among the plurality of processing rooms,
the container moving device carries out the preceding container from the processing room for the final process and, thereafter, the air conditioning device ventilates the processing room for the final process for the predetermined time and, then, the container moving device carries the container storing the different culture into the processing room for the final process.

3. The culture processing device according to claim 1, wherein
a group is formed by a plurality of containers storing a same culture,
when the containers of the same group are processed, the container moving device sequentially moves the plurality of containers from the processing room for the upstream process to the processing room for the downstream process,
when a container of a different group formed by a plurality of containers storing a culture different from a culture of a precedingly processed group is processed, the container moving device moves all containers of the preceding group from the processing room for the upstream process to the processing room for the downstream process adjacent to each other and, thereafter, the air conditioning device ventilates the processing room for the upstream process for the predetermined time and, then, the container moving device carries the container of the different group into the processing room for the upstream process.
